(19) United States
(12) Patent Application Publication
(10) Pub. No.: US 2021/0363478 A1
(43) Pub. Date: Nov. 25, 2021

(54) DEVICE AND METHOD FOR THE EXTRACTION OF STEM CELLS FROM ADIPOSE TISSUE

(71) Applicants: UNIVERSITA' DEGLI STUDI DI BARI ALDO MORO, BARI (IT); MASMEC S.P.A., MODUGNO (IT)

(72) Inventors: Francesco GIORGINO, BARI (IT); Sebastio PERRINI, BARI (IT); Pietro LARIZZA, MODUGNO (IT)

(21) Appl. No.: 17/325,920

(22) Filed: May 20, 2021

(30) Foreign Application Priority Data
May 21, 2020 (IT) 102020000011926

(51) Int. Cl. C12M 1/00; C12N 5/077; C12M 1/12

(52) U.S. Cl. CPC C12M 47/04; C12M 25/02; C12M 45/03; C12N 5/0653

(57) ABSTRACT

A device and method for the extraction of stem cells from adipose tissue wherein an adipose tissue dissolved in an isotonic solution is subjected to a washing operation and subsequent filtering step and to centrifugation operations to initiate three stem cell cultures thereby maximising in this manner the yield of the sample collected.

### CROSS-REFERENCE TO RELATED APPLICATIONS

This Patent Application claims priority from Italian Patent Application No. 102020000011926 filed on May 21, 2020, the entire disclosure of which is incorporated herein by reference.

### TECHNICAL FIELD

The present invention relates to a device and to a method for the extraction of stem cells from adipose tissue.

### BACKGROUND OF THE INVENTION

As is known, stem cells are primitive, unspecialised cells, with the ability to transform into different types of cells in the body through a process called cell differentiation. They have been studied by researchers to treat certain diseases by exploiting their ductility.

Stem cells can be collected from different sources, such as the umbilical cord, amniotic sac, blood, bone marrow, placenta, and adipose tissues.

Among the various tissues listed above, adipose tissue is easily accessible and therefore forms the most convenient extraction region.

This tissue contains many types of cells, including connective cells, stroma and a small percentage (around 10%) of stem cells.

For this reason, it is necessary to extract a large amount of tissue (typically 6-10 grams) to be able to proceed with extraction of the stem cells. However, in some particularly thin subjects, it is difficult to obtain this amount of tissue.

The object of the present invention is to produce a device and a method for the extraction of stem cells from a limited amount of adipose tissue, in particular an amount of adipose tissue of around 1 gram. In particular, the object of the present invention is to produce a device that operates completely automatically.

Moreover, the object of the present invention is to produce a device and a method for the extraction of stem cells that makes it possible to control the experimental variables currently dependent on the single operator and capable of obtaining, with greater probability, homogeneous cells from a phenotypic, functional and genetic point of view.

### SUMMARY OF THE INVENTION

The preceding object is achieved by the present invention, which relates to a device and to a method for the extraction of stem cells from adipose tissue as described in the appended claims.

### BRIEF DESCRIPTION OF THE DRAWINGS

The invention will now be illustrated with reference to the accompanying figures which represent a non-limiting embodiment thereof, wherein:

FIG. 1 illustrates, in a schematic view from above, a device produced according to the dictates of the present invention;

FIG. 2 illustrates a sequence of steps carried out by the present device; and

FIG. 3 illustrates a detail of the device of FIG. 1.

### DETAILED DESCRIPTION OF THE INVENTION

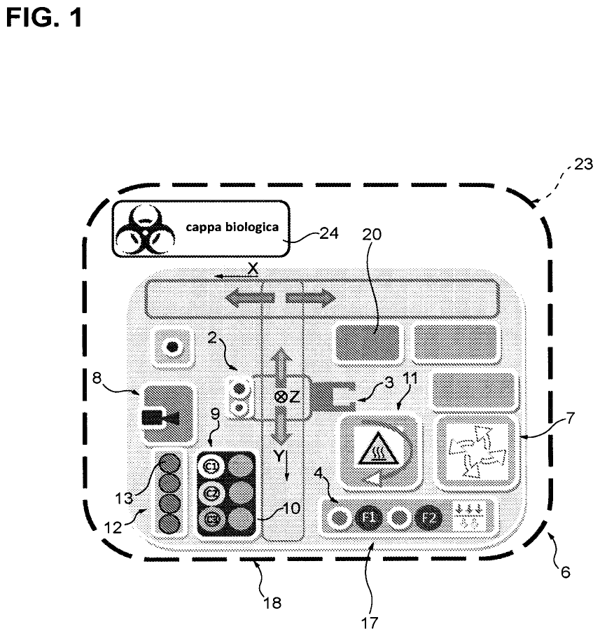

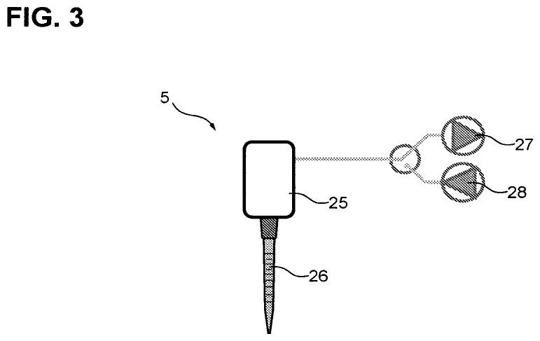

With reference to FIG. 1, the device 1 of the present invention is provided with a robotised handling system 2 (of a known type and represented schematically) wherein a gripping element (gripper) 3 that moves in three-dimensional space along three axes X, Y and Z under the thrust of actuators is designed to collect and move a container 4/a suction and discharge system 5 (FIG. 3) to move it between different units, which will be illustrated below. The suction/discharge system 5 is capable of managing volumes of fluid that exceed 3 ml and is also designed to aspirate and release high density fluids.

The device 1 comprises the following units: a filtration system 6 (of a known type), a heat stirrer 11 (of a known type), a centrifugation system 7 (of a known type), a viewing system 8 (of a known type), and a plurality of containers 9 for the cell culture arranged on a supporting structure (rack) 10, a rack 12 for reagents 13, a buffer station 17 in which the container 4 can be temporarily arranged.

There is provided a loading station 18 in which a container 4 to be subjected to treatment is arranged.

There is provided a single container 23 (illustrated schematically) provided with a biological hood 24 and configured to contain the robotised handling system 2, the filtration system 6, the stirrer 11, the centrifugation system 7, the culture devices, the reagents rack 12, the viewing system 8 and the buffer station 17.

With reference to FIG. 3, the suction and discharge system 5 comprises a container 25 provided, at one end thereof, with a tip 26 designed to aspirate/release fluids. The container 25 is connected with a suction system 27 to produce the vacuum in the container 25 and obtain suction of fluid in the tip 26 and with a system 28 designed to alternatively generate a pressure to produce the increase in pressure in the container 25 and obtain expulsion of the fluid contained in the tip 26.

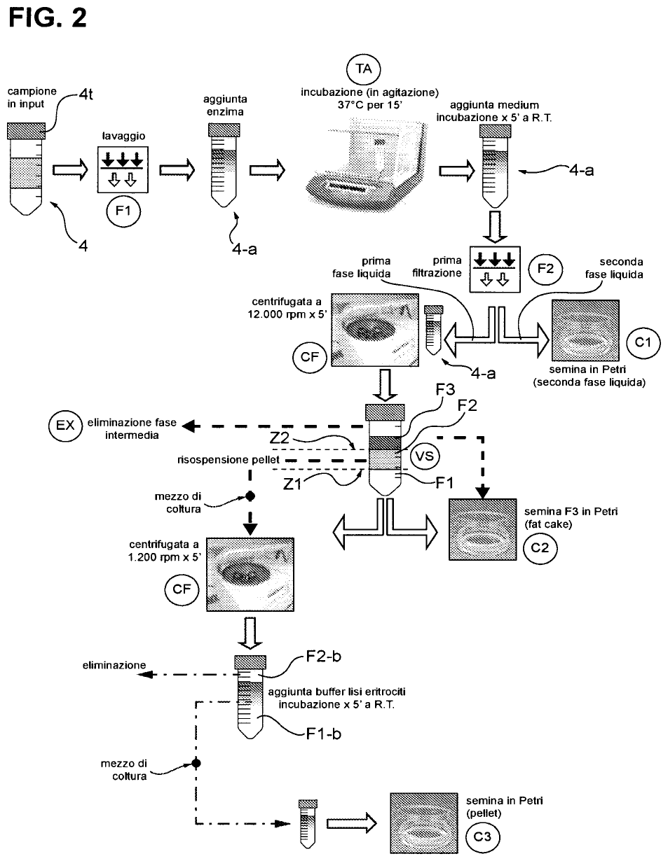

The operations of the device 1 are performed according to what is illustrated below in FIG. 2 based on controls imparted by an electronic control unit 20.

Advantageously, the adipose tissue collected from the patient is dissolved in an isotonic solution (input sample in FIG. 2) and contained in a graduated transparent tubular container 4 provided with a screw-on cap 4t. For example, the container known with the trade name Falcon 50, which can contain 50 ml of liquid, can be used. As is known, a solution is called isotonic when it has the same osmotic pressure, with respect to another solution from which it is separated through a semi-permeable membrane.

The container 4 is manually arranged on the loading station 18: subsequently, the electronic unit 20 automatically controls collection of the container 4 by means of the gripper 3 so that the container 4 is transferred to the filtration system 6, using 250 mcm nylon filters, where a first washing step with isotonic solutions is carried out by means of known techniques.

In this step, the lipoaspirate collected in the initial step is inserted (placed) on basket type nylon filters, in turn placed on Falcon tubes, and washed three times under gravity with isotonic solution.

Preferably, the washing liquid can be ejected with a high-pressure jet in order to facilitate removal of blood clots and tissue debris.

The lipoaspirate, purified of the blood clots and tissue debris, is transferred from the nylon filter support into a Falcon 4-a sterile container by means of the gripper 3 controlled by the control unit 20.

The electronic control unit 20 is subsequently designed to carry out a step in which an isotonic solution containing a predetermined amount of enzyme designed to start a cell disruption process is added to the lipoaspirate. This step is advantageously carried out by arranging the container 4 in the buffer station 17 and utilising the discharge suction system 5 carried by the gripper 3 and designed to collect the enzyme from the rack 12 of the reagents 13 and release the enzyme inside the container 4 (see FIG. 2; collagenase can be used, for example, as enzyme).

The electronic control unit 20 is designed to carry out a stirring and incubation step TA in which the solution of lipoaspirate and enzyme contained in the container 4 is pre-washed by the buffer station 17, supplied to the stirrer 11 where the solution of lipoaspirate and enzyme is stirred, thereby starting a chemical and mechanical breakdown process of the adipose tissue. The stirring operations take place, preferably but not exclusively, at 250 rpm and at a predetermined temperature of 37° C. corresponding to the temperature of the human body. This temperature is guaranteed by a thermoregulator (not illustrated).

The electronic unit 20 controls collection of the container 4-a from the stirrer 11 and re-transfers the container 4 to the filtration system 5 where a first filtering step is carried out, again by means of known techniques, such as using nylon filters.

A first microparticulate liquid phase containing the adipose stem cells is thus separated from a second semi-liquid phase of undigested adipose tissue.

The second semi-liquid phase of undigested adipose tissue is transferred by means of the gripper 3 into a first culture device C1 of a known type generally formed by a Petri plate or Petri capsule. This culture device C1 is arranged on the supporting rack 12 and is maintained in a controlled environment to obtain multiplication of the cells, thereby growing a first culture C1 of adipose stem cells.

The electronic unit 13 re-transfers the container 4-a to the centrifugation system 7 that receives the first microparticulate liquid phase and subjects it to a centrifugation operation CF producing a product that separates into three liquid phases and precisely:
a first denser liquid phase F1 (pellet) that contains mainly stem cells; this phase is arranged on the bottom of the vertically oriented container 4-a:
a second liquid phase F2 with intermediate density that contains mainly BSA, HEPES glucose, NaCl, KCl, CaCl₂ dissolved in water; this phase is arranged in the container 4-a on top of the first phase F1;
a third liquid phase F3 with lower density that contains, based on the findings of the inventors, stem cells and mature adipose cells (fat cake); this third phase is arranged in the container 4-a on top of the second phase F2.

The viewing system 8 detects an image of the three liquid phases arranged in the transparent container 4 and identifies on the image the separation zones Z1, Z2 between the first and second phases and the second and third phases, respectively. Knowing the separation zones Z1, Z2 allows the selective collection of the three phases, for example, by means of selective suction.

This suction is carried out using the suction and discharge system 5 carried by the gripper 3.

According to the present invention, the third liquid phase F3 (also called fat cake), after having been separated from the other phases, is transferred by means of the gripper 3 into a second culture device C2 of a known type generally formed by Petri plates or Petri capsules. This second culture device C2 is arranged on the supporting rack 12 and is maintained in a controlled environment to obtain multiplication of the stem cells thereby growing a second culture of adipose stem cells. Growth of the stem cells is controlled by means of observation, by the operator, with an optical microscope that is carried into position by the gripper 3. This microscope is associated with a viewing system for viewing images of the culture on the monitor.

After having eliminated the second liquid phase F2 with intermediate density (EX), a culture medium utilised for growth of the adipose stem cells is added to the first liquid phase F1 (pellet). The electronic unit 13 re-transfers the container containing the first liquid phase F1 (containing pellet+culture medium) to the centrifugation system 7 and subjects it to a centrifugation operation CF typically carried out at 1,2000 rpm for 5 minutes at room temperature producing a product that separates into two phases and precisely:
a further first denser liquid phase F1-b (pellet) that contains mainly stem cells; this phase is arranged on the bottom of the vertically oriented container 5; and
a further second liquid phase F2-b with intermediate density that contains culture medium; this phase is arranged in the container 5 on top of the further first phase F1-b.

By means of selective suction the further second liquid phase F2-b is eliminated and culture medium is added to the further first liquid phase F1-b for resuspension of the pellet. The suspension containing the further first phase F1-b and the culture medium is transferred by the gripper 3 into a third culture device C3 of a known type, generally formed by a Petri plate or Petri capsule. This third culture device is arranged on the supporting rack 12 and is maintained in a controlled environment to obtain multiplication of the stem cells thereby growing a third culture of adipose stem cells. Also in this case, growth of the stem cells is controlled by means of observation, by the operator, with an optical microscope that is carried into position by the gripper 3.

The novelty of the present invention is represented by initiation of several cultures (three) of stem cells thereby maximising the yield of the sample collected.

In fact, the inventors of the present patent application have discovered that also the first phase, represented by undigested adipose tissue and which is normally eliminated, contains a sufficient number of stem cells to initiate a cell culture. The procedure carried out by the device illustrated above is completely automatic, as the operator requires only to arrange the sample in the loading position, after which all the operations for processing the sample are carried out by the device 1 without any human intervention. The operator only provides for collection of the culture devices.

1. A device (1) for the extraction of stem cells from adipose tissue, wherein a robotised handling system (2) is provided with a gripper (3) that moves in three-dimensional space and is designed to collect and move a container (4)/a suction and discharge system (5) to move it between different units comprising at least a filtration system (6), a stirrer (11) and a centrifugation system (7);
an electronic control unit (20) is configured to control the performance of the following operations:
collecting by means of the gripper (3) a container (4) containing adipose tissue possibly dissolved in an isotonic solution and transferring this container (4) to the filtration system (6) where a washing step is carried out to eliminate blood clots and tissue debris;
adding to the adipose tissue subjected to washing an isotonic solution containing a predetermined amount of enzyme designed to start a cell disruption process;
supplying the solution of lipoaspirate and enzyme contained in the container (4) to the stirrer (11) to carry out a stirring step TA thereby starting a chemical and mechanical breakdown process of the adipose tissue;
transferring the container (4) to the filtration system (6) where a first filtering step is carried out, separating a first microparticulate liquid phase containing adipose stem cells and a second semi-liquid phase of undigested adipose tissue containing adipose stem cells;
transferring the second semi-liquid phase of undigested adipose tissue by means of the gripper (3) into a first culture device (C1) maintained in a controlled environment to obtain multiplication of the cells thereby growing a first culture (C1) of adipose stem cells;
transferring the container (4) containing the first microparticulate phase to the centrifugation system (7) that receives the first phase and subjects it to a centrifugation operation (CF) producing a product that separates inside the container into three phases and precisely:
a first denser liquid phase (F1) (pellet) that contains mainly stem cells—this first liquid phase (F1) is arranged on the bottom of a vertically oriented container;
a second liquid phase (F2) with intermediate density that contains mainly BSA, HEPES glucose, NaCl, KCl, CaCl₂ dissolved in water—this second liquid phase (F2) is arranged in the container on top of the first liquid phase (F1);
a third liquid phase (fat cake; F3) with lower density that contains stem cells and mature adipose cells—this third phase is arranged in the container on top of the second liquid phase (F2);
separating the third phase (F3) from the other phases by means of the suction and discharge system (5) carried by the gripper (3) and placing it in a second culture device C2 that is maintained in a controlled environment to obtain multiplication of the stem cells thereby growing a second cell culture (C2); and
separating the first phase (F1) from the other phases by means of the suction and discharge system and adding a culture medium to the first liquid phase (F1);
the electronic unit (13) is configured to transfer the container containing the first phase containing the culture medium to the centrifugation system (7) that subjects the first phase to a centrifugation operation (CF) producing a product that separates into two further liquid phases and precisely:
a further denser first phase (F1-b) (pellet) that contains mainly stem cells—this further first phase is arranged on the bottom of the vertically oriented container (5);
a further second phase (F2-b) with intermediate density that contains the culture medium—this further second phase is arranged in the container (5) on top of the first phase (F1);
the electronic unit is designed to control the selective suction of the second further phase (F2-b) that is eliminated and is designed to add a culture medium to the further first phase (F1-b) for resuspension of the pellet;
the electronic unit is designed to control the transfer by means of gripper of the suspension containing the pellet into a third culture device (C3) maintained in a controlled environment to obtain multiplication of the stem cells thereby growing a third culture of adipose stem cells.

2. The device according to claim 1, wherein there is provided a viewing system (8) configured to detect an image of the three phases arranged in the transparent container (4) and identifying on the image the separation zones Z1, Z2 between the first and the second phase and the second and the third phase, respectively; said electronic unit (20) being configured to control the selective collection of the three phases, for example by means of selective suction, based on the information on the separation zones.

3. The device according to claim 1, wherein there is provided a single container (23) provided with a biological hood (24) and configured to contain the robotised handling system (2), the filtration system (6), the stirrer (11), the centrifugation system (7) and the culture devices.

4. The device according to claim 1, wherein said suction and discharge system (5) comprises a container (25) provided, at one end thereof, with a tip (26) configured to aspirate/release fluids; said container (25) is connected with a suction system (27) to produce the vacuum in the container (25) and obtain suction of fluid in the tip (26) and with a system (28) designed to generate a pressure to produce the increase in pressure in the container (25) and obtain expulsion of the fluid contained in the tip (26).

5. A method for the extraction of stem cells from adipose tissue comprising the following operations:
collecting a container (4) containing adipose tissue possibly dissolved in an isotonic solution and transferring this container (4) onto a filter to carry out a washing step of the lipoaspirate;
adding to the lipoaspirate subjected to washing a solution containing a predetermined amount of enzyme designed to start a cell disruption process;
supplying the solution of lipoaspirate and enzyme contained in the container (4-a) to a stirrer (11) to carry out a stirring step TA thereby starting a chemical and mechanical breakdown process of the adipose tissue;
transferring the container (4) to the filtration system (6) where a first filtering phase is carried out, separating a first microparticulate liquid phase containing adipose stem cells and a second semi-liquid phase containing undigested adipose tissue;
supplying the second semi-liquid phase of undigested adipose tissue to a first culture device (C1) maintained in a controlled environment to obtain multiplication of the cells thereby growing a first culture (C1) of adipose stem cells;
transferring the container (4) containing the first microparticulate phase to the centrifugation system (7) that receives the first microparticulate liquid phase and subjects it to a centrifugation operation (CF) producing a product that separates inside the container into three phases and precisely:
a first denser liquid phase (F1) (pellet) that contains mainly stem cells—this phase is arranged on the bottom of a vertically oriented container:
a second liquid phase (F2) with intermediate density that contains mainly BSA, HEPES glucose, NaCl, KCl, CaCl₂ dissolved in water—this phase is arranged in the container on top of the first phase (F1);
a third liquid phase (fat cake; F3) with lower density that contains stem cells and mature adipocytes—this third liquid phase (F3) is arranged in the container on top of the second liquid phase (F2);
separating the third liquid phase (F3) from the other phases and placing it in a second culture device (C2) that is maintained in a controlled environment to obtain multiplication of the stem cells thereby growing a second cell culture (C2); and
separating the first liquid phase (F1) from the other phases and adding a culture medium to the first liquid phase (F1);
subjecting the first liquid phase (F1) containing the culture medium to a centrifugation operation (CF) producing a product that separates into two further phases and precisely:
a further first denser liquid phase (F1-b) that contains mainly stem cells—this phase is arranged on the bottom of the vertically oriented container (5);
a further second liquid phase (F2-b) with intermediate density that contains the culture medium—this further second phase (F2-b) is arranged in the container (5) on top of the further first phase (F1-b);
eliminating by means of selective suction the further second liquid phase (F2-b) and adding a culture medium to the further first phase (F1-b) for resuspension of the pellet;
transferring the suspension containing the pellet into a third culture device (C3) maintained in a controlled environment to obtain multiplication of the stem cells thereby growing a third culture (C3) of adipose stem cells.

6. The method according to claim 5, wherein there is provided the step of detecting an image of the three phases arranged in the transparent container (4) and identifying on the image the separation zones Z1, Z2 between the first and the second phase and the second and the third phase, respectively; selectively collecting the three phases, for example by means of selective suction, based on the information on the separation zones.
